(19) United States
(12) Patent Application Publication
(10) Pub. No.: US 2015/0216702 A1
(43) Pub. Date: Aug. 6, 2015

(54) ANKLE BRACE

(71) Applicant: SHOCK DOCTOR, INC., Plymouth, MN (US)

(72) Inventors: William Best, Orono, MN (US); Bastien Jourde, Montreal (CA); Thierry Patelle, Montreal (CA)

(73) Assignee: Shock Doctor, Inc., Minnetonka, MN (US)

(21) Appl. No.: 14/428,925

(22) PCT Filed: Sep. 17, 2013
(86) PCT No.: PCT/US13/60152

§ 371 Date: Mar. 17, 2015

Related U.S. Application Data
(60) Provisional application No. 61/702,081, filed on Sep. 17, 2012.

(51) Int. Cl. A61F 5/01

(52) U.S. Cl. CPC A61F 5/0127; A61F 2250/001

(57) ABSTRACT

A reversible ankle brace is easy to use, is comfortable for the wearer and is reversible. Accordingly, a wearer may use the inventive ankle brace on either their left foot, to support their left ankle, or their right foot, to support their right ankle, without having to purchase more than one ankle brace.

### RELATED APPLICATIONS

This application is a U.S. National Stage Entry under 35 U.S.C. §371 of International Application No. PCT/US2013/060152, filed Sep. 17, 2013, which claims the benefit of U.S. Provisional Application Ser. No. 61/702,081, filed Sep. 17, 2012, entitled ANKLE BRACE, which are incorporated by reference herein in their entirety.

### TECHNICAL FIELD

The disclosure pertains generally to protective and rehabilitative gear and more particularly to an ankle brace.

### BACKGROUND

Each year, many people, both athletes and non-athletes, suffer ankle injuries. In some cases, athletes wrap their ankles with adhesive tape in an attempt to prevent ankle injuries and/or to support their ankles after an injury has occurred. In many instances, athletes and others use ankle braces to protect and/or to rehabilitate their ankles.

While a variety of ankle braces are known, there is a desire for continued improvement in the performance and comfort of known ankle braces. There is a desire for an ankle brace that is easy to use and comfortable for the wearer.

### SUMMARY

In some embodiments, the invention pertains to an ankle brace that is easy to use, is comfortable for the wearer and is reversible, i.e., a single ankle brace may be used on either the wearer's left foot or right foot, depending on where the injury is. A reversible ankle brace means that a wearer can use the brace again, even if, for example, they injure their other ankle

Accordingly, in some embodiments, the invention pertains to a reversible ankle brace that includes a stirrup having a base plate and first and second stirrup arms. A first sideplate is secured to the first stirrup arm at a first pivot point and a second sideplate is secured to the second stirrup arm at a second pivot point. First and second elongate slots are formed in the outer surfaces of the first and second sideplates. A first pad is releasably securable to an inner surface of the first sideplate and a second pad is releasably securable to an inner surface of the second sideplate. A first horizontal strap is releasably securable about an exterior of the first pad and the second pad and secures an upper portion of the ankle brace. A second horizontal strap extends through the first and second elongate slots and secures together an intermediate portion of the ankle brace.

In some embodiments, the reversible ankle brace further includes a cross strap. The cross strap is configured to fit through a pair of slots in the base plate, cross a first time on a back side of the ankle brace and cross a second time on a front side of the ankle brace. In some instances, the cross strap has a first end and a second end, and the first and second ends are adapted to be releasably securable to the first horizontal strap after crossing the first time behind the ankle brace and crossing the second time in front of the ankle brace.

In some embodiments, the invention pertains to a method of adjusting and putting a reversible ankle brace on a wearer. The method includes selecting a pair of slots that extend through a base plate of the reversible ankle brace in accordance with whether the ankle brace will be used on the wearer's left foot or their right foot. A cross strap is extended through the selected pair of slots such that the cross strap has a first end extending from a first side of the base plate and a second end extending from a second side of the base plate. After the wearer's foot is positioned within the reversible ankle brace, a first horizontal strap that extends around an exterior of the reversible ankle brace is secured in place in order to secure together an upper portion of the reversible ankle brace. A second horizontal strap that extends around an exterior of the reversible ankle brace is secured in place in order to secure together an intermediate portion of the reversible ankle brace. The first and second ends of the cross strap are extended inside the first and second side pads, the cross strap passing over either side of the wearer's foot and then wrapping behind the wearer's calf, the first and second ends of the cross strap crossing each other proximate the wearer's calf. The first and second ends of the cross strap are wrapped in front of the wearer's shin, the first and second ends of the cross strap crossing each other again proximate the wearer's shin. The first and second ends of the cross strap are secured to either side of one of the first and second horizontal straps.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE FIGURES

FIG. 1 is a perspective view of an ankle brace in accordance with embodiments of the invention, shown on a foot.

FIG. 2 is a perspective view of the ankle brace of FIG. 1, shown without the foot.

FIG. 3 is a front perspective view of another ankle brace in accordance with embodiments of the invention, shown on a foot.

FIG. 4 is a front perspective view of the ankle brace of FIG. 3, shown without the foot.

FIG. 5 is a rear perspective view of the ankle brace of FIG. 3, shown without the foot.

FIG. 6 is a front perspective view of a portion of the ankle brace of FIG. 1, shown with side pads attached.

FIG. 7 is a front view of the ankle brace of FIG. 6.

FIG. 8 is a front perspective view of a portion of the ankle brace of FIG. 7, shown without side pads.

FIG. 9 is a front view of the ankle brace of FIG. 8.

FIG. 10 is a medial side view of the ankle brace of FIG. 6.

FIG. 11 is a lateral side view of the ankle brace of FIG. 6.

FIG. 12 is an exploded schematic view of the ankle brace of FIG. 1.

FIG. 13 is an exploded schematic view of the ankle brace of FIG. 3.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

Various embodiments relate to a reversible ankle brace that is easy to use, is comfortable for the wearer and is reversible. Accordingly, a wearer may use the inventive ankle brace on either their left foot, to support their left ankle, or their right foot, to support their right ankle, without having to purchase more than one ankle brace. Though ankle braces according to various embodiments include the features and/or achieve the foregoing advantages, alternative or additional features and advantages are contemplated.

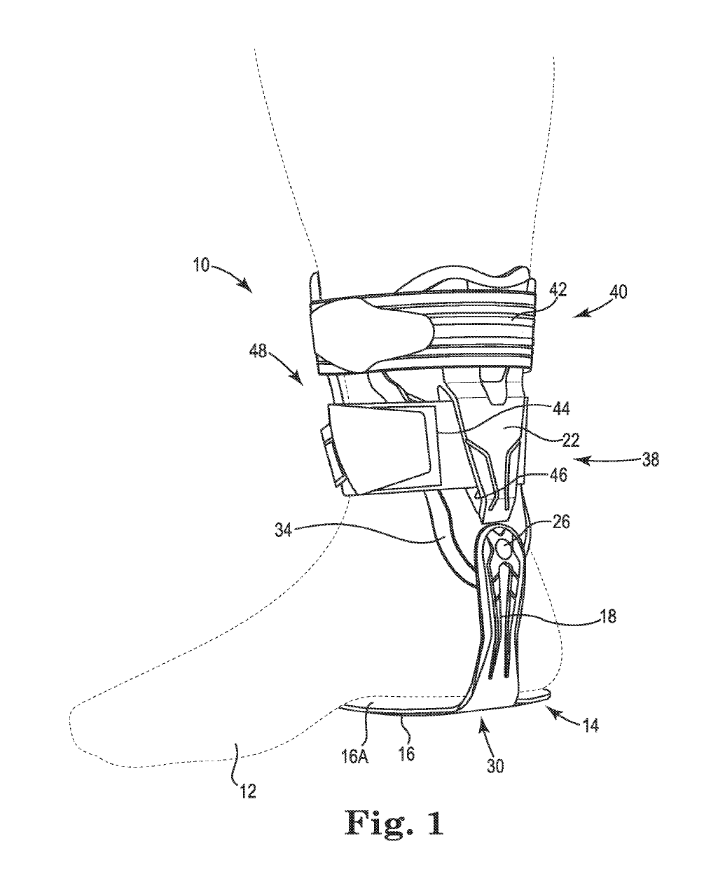

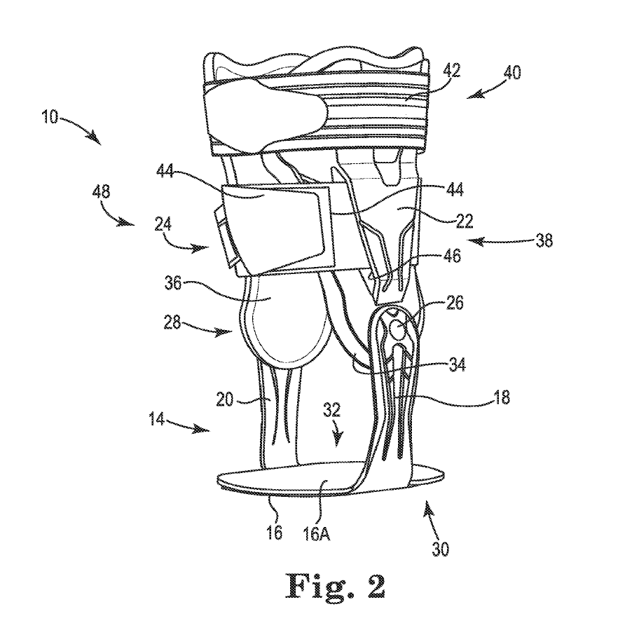

FIGS. 1 and 2 provide perspective views of an ankle brace 10. In FIG. 1, the ankle brace 10 is shown on a wearer's foot 12 while FIG. 2 provides a similar view without the foot 12, in order to show additional details of the ankle brace 10.

The ankle brace 10 includes a stirrup 14 having a base plate 16, a first stirrup arm 18 and a second stirrup arm 20. As illustrated, the first stirrup arm 18 is approximately centered along a first side 30 of the stirrup 14 and the second stirrup arm 20 is approximately centered along a second side 32 of the stirrup 14. In some embodiments, centering the first stirrup arm 18 and the second stirrup arm 20 facilitates being able to reverse the ankle brace 10 for use on either a left ankle or a right ankle. Optionally, the stirrup 14 is integrally molded as a unitary piece, including the base plate 16, the first stirrup arm 18 and the second stirrup arm 20. The stirrup 14 is optionally formed of a relatively rigid polymeric material. In some embodiments, a soft foam pad 16A is disposed over the base plate 16 for the wearer's comfort.

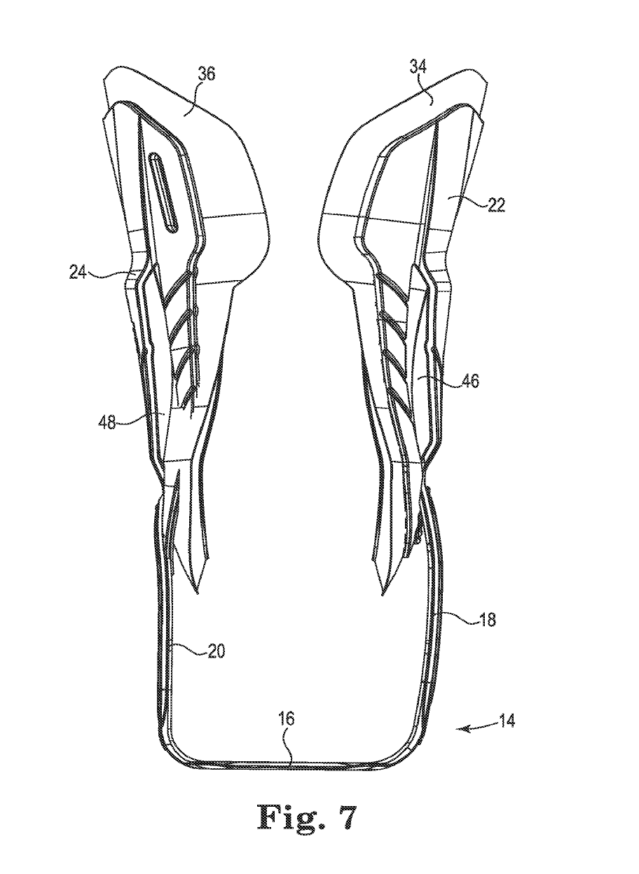

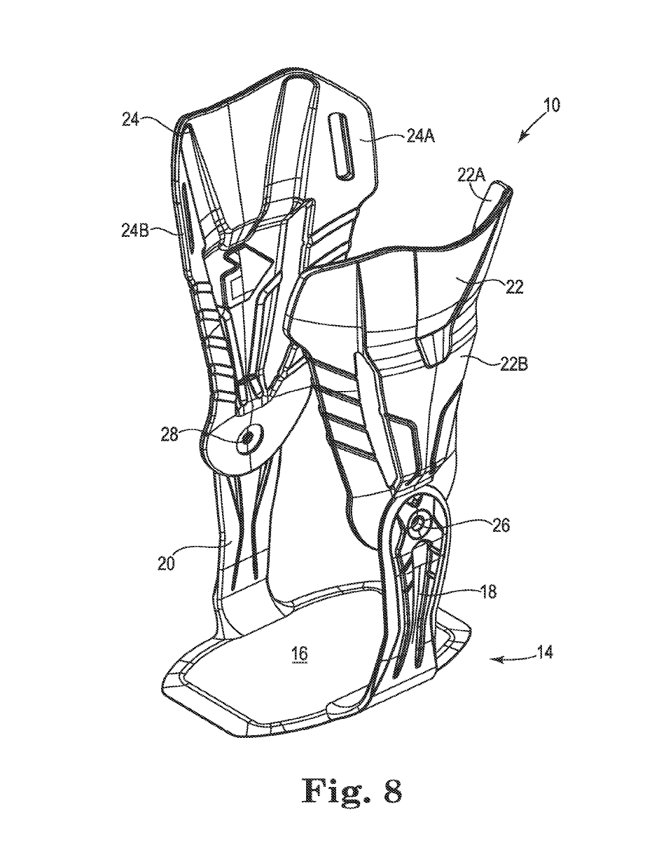

In some embodiments, the ankle brace 10 is configured to wear with one of the first stirrup arm 18 and the second stirrup arm 20 on the medial (inside) side of the wearer's ankle and have the other of the first stirrup arm 18 and the second stirrup arm 20 on the lateral (outside) side of the wearer's ankle In some instances, one of the first stirrup arm 18 and the second stirrup arm 20 has a slightly different length or profile than the other. For example, and with particular reference to FIGS. 7 and 8, the first stirrup arm 18 is longer than the second stirrup arm 20 and has a more pronounced curve. The second stirrup arm 20 is straighter than the first stirrup arm 18, and has a smaller curve near a top thereof.

First and second sideplates 22 and 24 are attached to the first and second stirrup arms 18, 20, respectively, at a first pivot point 26 and a second pivot point 28. In some embodiments, the stirrup 14 is symmetric about a medial, vertical plane (not illustrated, for clarity) that extends through the first pivot point 26 and the second pivot point 28 and that is perpendicular to the base plate 16. It will be appreciated that the first sideplate 22 may be permitted to pivot forwards and backwards relative to the first stirrup arm 18 while being constrained against relative movement in a side to side fashion. Similarly, the second sideplate 24 may be permitted to pivot forwards and backwards relative to the second stirrup arm 20 while being constrained against relative movement in a side to side fashion. The first sideplate 22 and the second sideplate 24 are optionally formed of a relatively rigid polymeric material.

A first pad 34 is releasably securable to the first sideplate 22 and a second pad 36 is releasably securable to the second sideplate 24. In some instances, a hook-and-loop fastening system, commercially known as VELCRO, is used to secure the first pad 34 to the first sideplate 22 and to secure the second pad 36 to the second sideplate 24. Optionally, the first and second pads 34, 36 are secured using alternate fashioning techniques such as snaps or pressure sensitive adhesives. The first pad 34 and the second pad 36 are optionally formed of a soft polymeric material such as a foam.

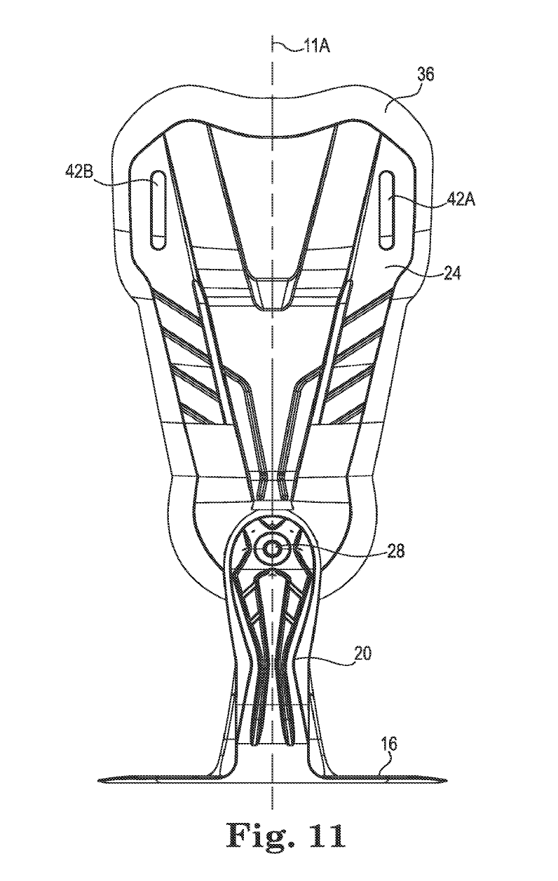

In some instances, the ankle brace 10 includes an intermediate portion 38 and an upper portion 40. As illustrated, a first horizontal strap 42 extends around an exterior of the upper portion 40 of the ankle brace 10. It will be appreciated that the first horizontal strap 42, if appropriately tightened up, will secure together the upper portion 40 of the ankle brace 10. In some cases (see FIG. 11) the first horizontal strap 42 is releasably secured to either the first sideplate 22 or the second sideplate 24 in order to appropriately locate the first horizontal strap 42 relative to the ankle brace 10. The first horizontal strap 42 is dimensioned to fit at least once around the upper portion 40 of the ankle brace 10. Optionally, the first horizontal strap 42 is dimensioned to fit more than once around the upper portion 40 of the ankle brace 10. The first horizontal strap 42 is configured to be securable to itself, such as by using hook-and-loop fasteners and may be formed of any elastomeric material.

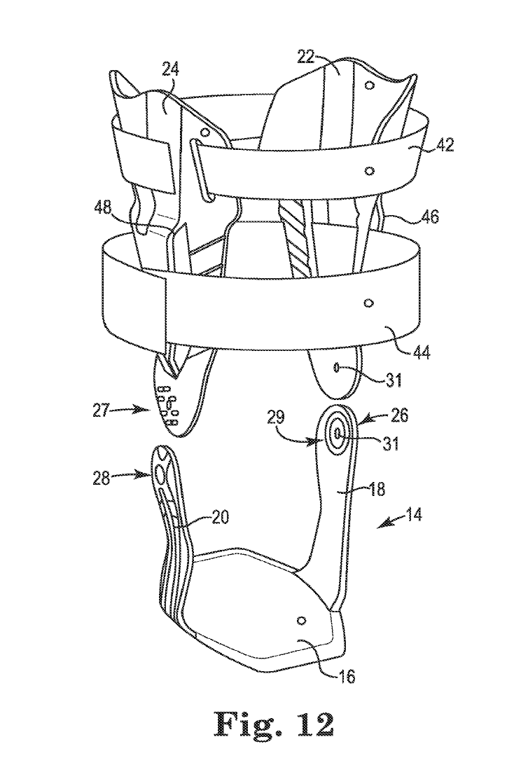

A second horizontal strap 44 extends around an exterior of the intermediate portion 38 of the ankle brace 10. As illustrated, the first sideplate 22 includes a first elongate slot 46. The second sideplate 24 includes a second elongate slot 48 (visible in FIG. 12). In some instances, the second horizontal strap 44 is extended through the first elongate slot 46 and the second elongate slot 48 in order to help guide the placement of the second horizontal strap 44. Optionally, the first and second elongate slots 46, 48 also provide guidance to a cross strap, as will be discussed with respect to subsequent Figures.

Optionally, the second horizontal strap 44 may be releasably secured (instead of or in addition to the first and second elongate slots 46, 48) to either the first sideplate 22 and the second side plate 24 in order to appropriately locate the second horizontal strap 44 relative to the ankle brace 10. The second horizontal strap 44 is dimensioned to fit at least once around the intermediate portion 38 of the ankle brace 10. Optionally, the second horizontal strap 44 is dimensioned to fit more than once around the intermediate portion 38 of the ankle brace 10. The second horizontal strap 44 is configured to be securable to itself, such as by using hook-and-loop fasteners and may be formed of any elastomeric material.

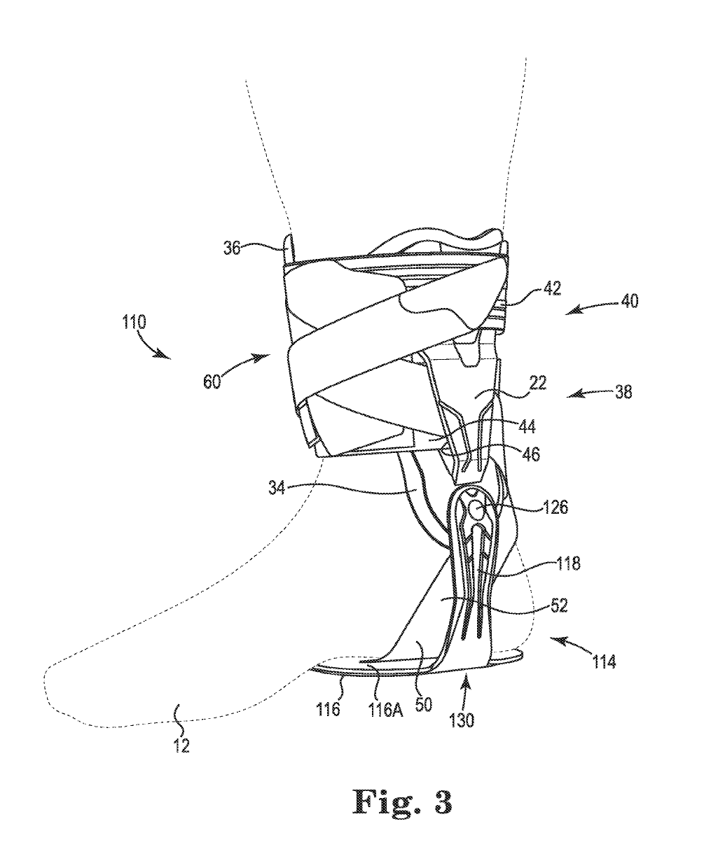

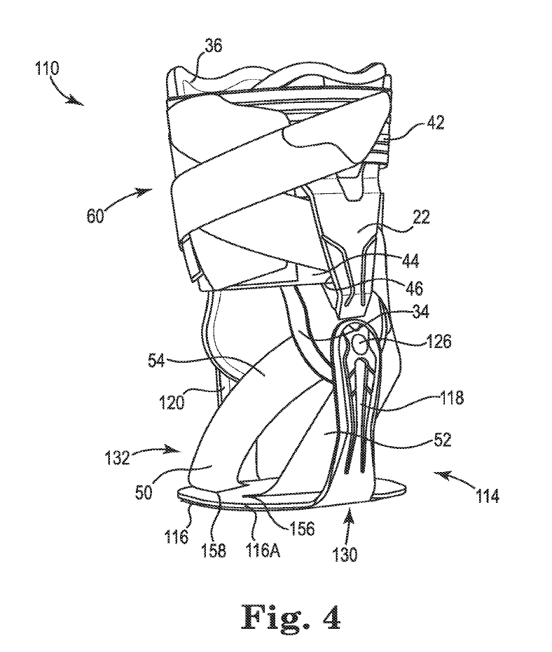

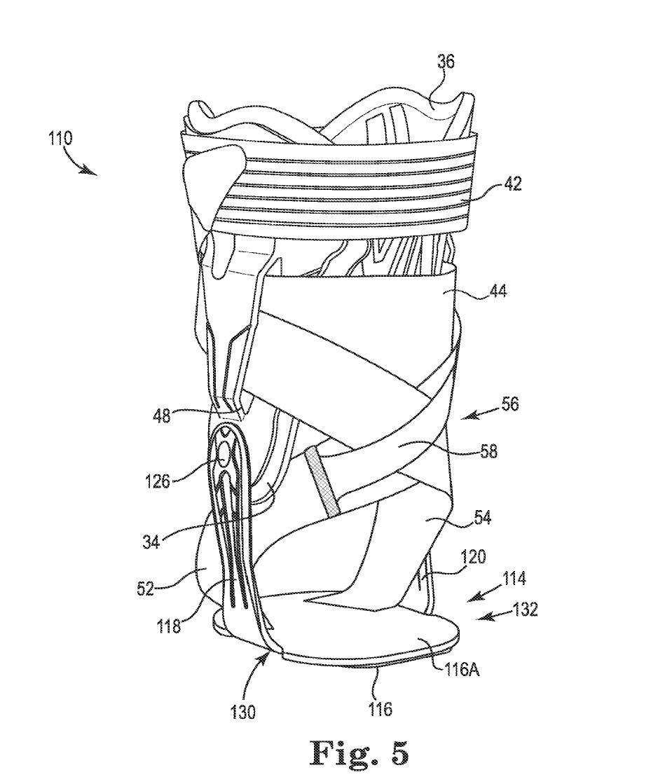

FIGS. 3, 4 and 5 provides prospective views of an ankle brace 110. In FIG. 3, the ankle brace 110 is shown on a wearer's foot 12 while FIGS. 4 and 5 provide a similar view without the foot 12, in order to show additional details of the ankle brace 110. According to some embodiments, the ankle brace 110 shares some common features with the ankle brace 10, and similar reference numbers are used to denote similar features. Various features that are the same between the ankle brace 10 and the ankle brace 110 are not be described again with specific reference to the ankle brace 110.

The ankle brace 110 primarily differs from the ankle brace 10 by including a cross strap 50. The ankle brace 110 includes a stirrup 114 that is configured to accommodate the cross strap 50. The stirrup 114 has a base plate 116, a first stirrup arm 118 and a second stirrup arm 120. As illustrated, the first stirrup arm 118 is approximately centered along a first side 130 of the stirrup 114 and the second stirrup arm 120 is approximately centered along a second side 132 of the stirrup 14. Optionally, the stirrup 114 is integrally molded as a unitary piece, including the base plate 116, the first stirrup arm 118 and the second stirrup arm 120. In some embodiments, a soft foam pad 116A is disposed over the base plate 116 for the wearer's comfort, or other purposes as desired.

First and second sideplates 22 and 24 are attached to the first and second stirrup arms 118, 120, respectively, at a first pivot point 126 and a second pivot point 128. In some embodiments, the stirrup 114 is symmetric about a medial, vertical plane (not illustrated) that extends through the first pivot point 126 and the second pivot point 128 and that is perpendicular to the base plate 116. It will be appreciated that the first sideplate 22 may be permitted to pivot forwards and backwards relative to the first stirrup arm 118 while being constrained against relative movement in a side to side fashion. Similarly, the second sideplate 24 may be permitted to pivot forwards and backwards relative to the second stirrup arm 120 while being constrained against relative movement in a side to side fashion.

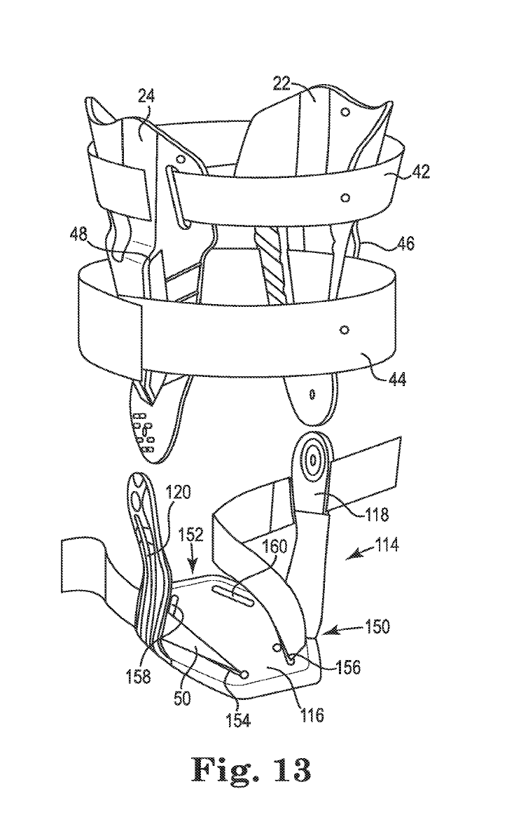

The base plate 116 includes a first pair 150 of slots and a second pair 152 (visible in FIG. 13) of slots. The first pair 150 of slots includes a slot 154 and a slot 156. The second pair 152 of slots includes a slot 158 and a slot 160. It will be appreciated that a wearer may select between the first pair 150 of slots and the second pair 152 of slots depending on whether they desire to use the ankle brace 110 on their left ankle or their right ankle For example, when worn on the right ankle, typically the first pair of slots 150 is selected.

In some embodiments, the wearer may prefer to have one of the first stirrup arm 118 and the second stirrup arm 120 on the medial (inside) side of their ankle and have the other of the first stirrup arm 118 and the second stirrup arm 120 on the lateral (outside) side of their ankle. In some instances, one of the first stirrup arm 118 and the second stirrup arm 120 may have a slightly different length or profile than the other, where the first stirrup arm 118 would typically be worn on the inside of the ankle.

Optionally, the cross strap 50 is used to provide improved heel retention within the ankle brace 110. The cross strap 50 may be formed of any elastomeric material. In some instances, the cross strap 50 is a single strap, i.e., the first end 52 and the second end 54 are part of a unitary cross strap 50. The cross strap 50 extends from either the first pair 150 of slots or the second pair 152 of slots, and may be considered as being divided into a first end 52 and a second end 54. As illustrated in FIGS. 3 to 5, the first end 52 and the second end 54 extend up from the base plate 116 and pass inside the first stirrup arm 118 and the second stirrup arm 120, respectively, and wrap along either side of the wearer's foot 12.

As shown in FIG. 5, the first end 52 and the second end 54 of the cross strap 50 intersect at a position 56 that would be behind the wearer's calf. Optionally, the first end 52 includes a slot 58 through which the second end 54 may pass. It will be appreciated that the second end 54 is free to move with respect to the first end 52 at the position 56. The cross strap 50 can be further wrapped such that the first end 52 passes through the second elongate slot 48 and the second end 54 passes through the first elongate slot 46 (not visible in FIG. 5). The cross strap 50 can be further wrapped such that the first end 52 and the second end 54 intersect at a position 60 that would be adjacent the wearer's shin. The second end 54 is free to move with respect to the first end 52 at the position 60. Optionally, the first end 52 and the second end 54 continue on and are releasably secured to the first horizontal band 42.

In an illustrative embodiment, and in order to adjust and wear the ankle brace 110, a user may select a pair 150, 152 of slots within the base plate 116, depending on which foot/ankle they wish to use the ankle brace 110 with. The wearer can extend the cross strap 50 through the selected pair 150, 152 of slots such that the cross strap 50 has a first end 52 extending from a first side of the base plate 116 and a second end 54 extending from a second side of the base plate 116.

The wearer would then place their foot within the ankle brace 110, and then would secure the first horizontal strap 42 that extends around an exterior of the ankle brace 110 in order to secure together an upper portion 40 of the ankle brace 110. The wearer would then secure the second horizontal strap 44 that extends around an exterior of the ankle brace 110 in order to secure together an intermediate portion 38 of the ankle brace 110. It will be appreciated that the first and second horizontal straps 42, 44 may be secured in place in either order, i.e., in some cases, the wearer may decide to secure the second horizontal strap 44 in place before securing in place the first horizontal strap 42.

Next, the wearer would extend the first and second ends 52, 54 of the cross strap 50 inside the first and second side pads 34, 36, the cross strap 50 passing over either side of the wearer's foot 12 and then wrapping behind the wearer's calf such that the first and second ends 52, 54 of the cross strap cross each other proximate the wearer's calf. The wearer would then wrap the first and second ends 52, 54 of the cross strap 50 in front of the wearer's shin such that the first and second ends 52, 54 of the cross strap 50 cross each other again proximate the wearer's shin. The wearer would then secure the first and second ends 52, 54 of the cross strap 50 to either side of one of the first and second horizontal straps 42, 44.

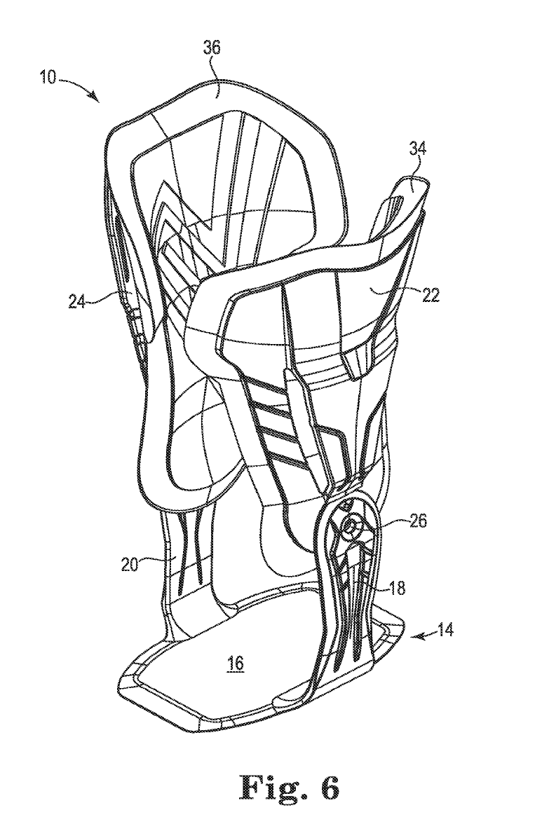

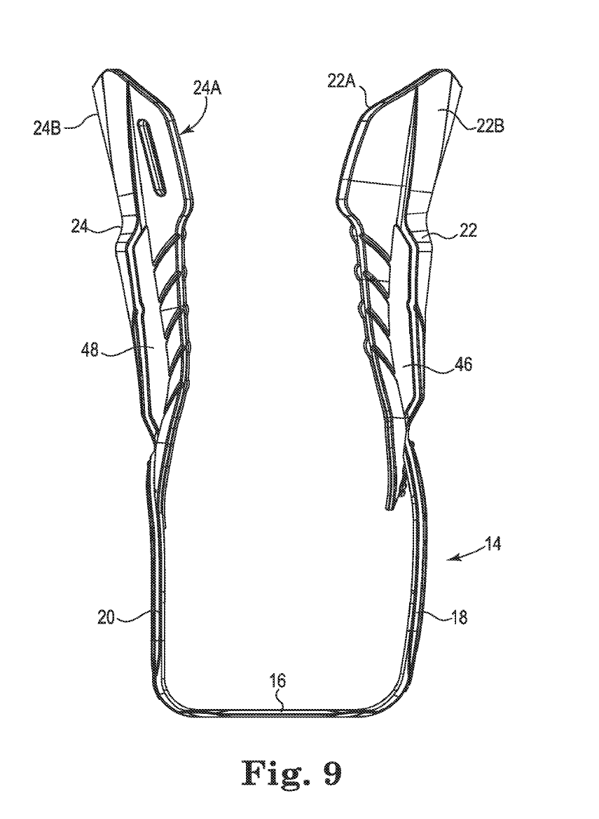

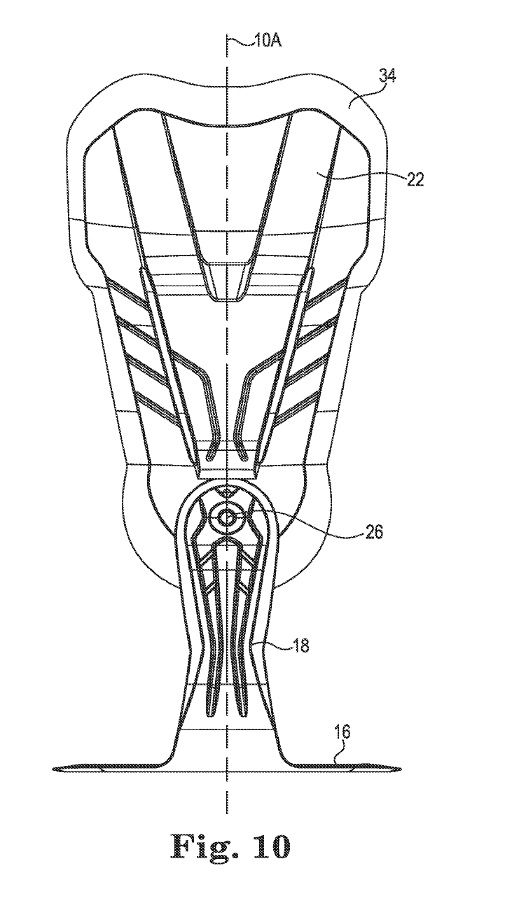

FIGS. 6 through 11 provide further views of elements of the ankle brace 10 already discussed. In particular, FIG. 6 is a front perspective view of a portion of the ankle brace of FIG. 1, shown with side pads attached, while FIG. 7 is a front view of the ankle brace of FIG. 6. FIG. 8 is a front perspective view of a portion of the ankle brace of FIG. 7, shown without side pads. FIG. 9 is a front view of the ankle brace of FIG. 8. FIG. 10 is a medial side view of the ankle brace of FIG. 6 and FIG. 11 is a lateral side view of the ankle brace of FIG. 6. It will be appreciated that the views of ankle brace 10 provided in FIGS. 6 through 11 are, with the exception of the stirrup, substantially similar to the ankle brace 110.

With reference to FIG. 8, it can be seen that the first sideplate 22 includes an inner surface 22A and an outer surface 22B and that the second sideplate 24 includes an inner surface 24A and an outer surface 24B. The first pad 34 may be releasably securable to the inner surface 22A of the first sideplate 22. The second pad 36 may be releasably securable to the inner surface 24A of the second sideplate 24. The first elongate slot 46 may be formed within the outer surface 22B of the first sideplate 22 and the second elongate slot 48 may be formed within the outer surface 24B of the second sideplate 22.

FIG. 10 is a medial side view of the ankle brace 10 and illustrates that the first stirrup arm 18 is centrally disposed along a side of the base plate 16 and that the first sideplate 22 is symmetric about a vertical midline 10A. FIG. 11 is a lateral side view of the ankle brace 10 and illustrates that the second stirrup arm 20 is centrally located along a side of the base plate 16 and further illustrates that the second sideplate 22 is symmetric about a vertical mid-line 11A. FIG. 11 also illustrates that the second sideplate 22 optionally includes slots 42A and 42B. The slots 42A and 42B, if present, are used to releasably secure the first horizontal strap 42 in place relative to the second sideplate 22 in order to facilitate wrapping the first horizontal strap 42 around the ankle brace 10.

FIGS. 12 and 13 are schematic illustrations of portions of the ankle brace 10 and the ankle brace 110, respectively, and provide further details regarding the first pivot point 26 and the second pivot point 28. While FIGS. 12 and 13 only show the outer surface 24B of the second sideplate 24 and the inner surface 22A of the first sideplate 22, it will be appreciated that these elements are symmetric, and thus features shown on the outer surface 24B of the second sideplate 22 are duplicated on the outer surface 22B of the first sideplate 22, and features shown on the inner surface 22A of the first sideplate 22 are duplicated on the inner surface 24A of the second sideplate 24.

Each sideplate 22, 24 includes a series of protrusions 27 that fit into a corresponding circular groove 29 that is formed into each stirrup arm 18, 20 (and 118, 120). It will be appreciated that the combination of protrusions 27 on the sideplates 22, 24 and corresponding circular grooves 29 on the stirrup arms 18, 20 facilitate and guide relative pivoting between the sideplate 22, 24 and the stirrup arm 18, 20. Each pivot point 26, 28 also includes an aperture 31 through which a rivet or similar fastener may be disposed.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A reversible ankle brace adapted for reversible wear on a left ankle or a right ankle, the ankle brace comprising:
a stirrup including a base plate, a first stirrup arm and a second stirrup arm;
a first sideplate secured to the first stirrup arm at a first pivot point, the first sideplate including a first inner surface and a first outer surface, a first elongate slot formed in the first outer surface;
a second sideplate secured to the second stirrup arm at a second pivot point, the second sideplate including a second inner surface and a second outer surface, a second elongate slot formed in the second outer surface;
a first pad releasably securable to the first inner surface;
a second pad releasably securable to the second inner surface;
a first horizontal strap releasably securable about an exterior of the first pad and the second pad, the first horizontal strap securing together an upper portion of the ankle brace; and
a second horizontal strap extending through the first elongate slot and the second elongate slot, the second horizontal strap releasably securable to itself, the second horizontal strap securing together an intermediate portion of the ankle brace.

2. The reversible ankle brace of claim 1, wherein the stirrup has a first side and a second side, the first stirrup arm being approximately centered along the first side and the second stirrup arm being approximately centered along the second side.

3. The reversible ankle brace of claim 1 or claim 2, wherein the base plate, the first stirrup arm and the second stirrup arm are integrally molded as a unitary piece.

4. The reversible ankle brace of any of claims 1 to 3, further comprising a foam pad adapted to fit onto the base plate.

5. The reversible ankle brace of any of claims 1 to 4, further comprising a third strap member.

6. The reversible ankle brace of claim 5, wherein the base plate includes a first pair of slots for accommodating the third strap member when the ankle brace is configured for use on a right ankle and a second pair of slots for accommodating the third strap member when the ankle brace is configured for use on a left ankle.

7. The reversible ankle brace of claim 6, wherein the third strap member is configured to fit through one of the pair of slots, cross a first time on a back side of the ankle brace and cross a second time on a front side of the ankle brace.

8. The reversible ankle brace of claim 7, wherein the third strap member has a first end and a second end, and the first and second ends are adapted to be releasably securable to the first horizontal strap after crossing the first time behind the ankle brace and crossing the second time in front of the ankle brace.

9. The reversible ankle brace of any of claims 1 to 8, wherein the first side member is secured to the first stirrup arm at the first pivot point such that the first sideplate can pivot towards a front of the stirrup and towards a back of the stirrup but is constrained from moving side to side.

10. The reversible ankle brace of any of claims 1 to 9, wherein the second side member is secured to the second stirrup arm at the second pivot point such that the second sideplate can pivot towards a front of the stirrup and towards a back of the stirrup but is constrained from moving side to side.

11. The reversible ankle brace of any of claims 1 to 10, wherein the first sideplate is symmetric about a vertical mid-line.

12. The reversible ankle brace of any of claims 1 to 11, wherein the second sideplate is symmetric about a vertical mid-line.

13. The reversible ankle brace of any of claims 1 to 12, wherein the stirrup is symmetric about a plane that is drawn through the first pivot point and the second pivot point and that is perpendicular to the base plate.

14. A method of adjusting and putting a reversible ankle brace on a wearer, the reversible ankle brace including first and second side pads, the method comprising steps of:
selecting a pair of slots through a base plate of the reversible ankle brace in accordance with whether the ankle brace is to be used on a left foot or a right foot;
extending a cross strap through the selected pair of slots such that the cross strap has a first end extending from a first side of the base plate and a second end extending from a second side of the base plate;
placing the wearer's foot within the reversible ankle brace;
securing a first horizontal strap that extends around an exterior of the reversible ankle brace, the first horizontal strap securing together an upper portion of the reversible ankle brace;
securing a second horizontal strap that extends around an exterior of the reversible ankle brace, the second horizontal strap securing together an intermediate portion of the reversible ankle brace;
extending the first and second ends of the cross strap inside the first and second side pads, the cross strap passing over either side of the wearer's foot and then wrapping behind the wearer's calf, the first and second ends of the cross strap crossing each other proximate the wearer's calf;
wrapping the first and second ends of the cross strap in front of the wearer's shin, the first and second ends of the cross strap crossing each other again proximate the wearer's shin; and
securing the first and second ends of the cross strap to either side of one of the first and second horizontal straps.

15. The method of claim 14, wherein the reversible ankle brace further comprises a first sideplate and a second sideplate, the first and second pads reversibly securable to the first and second sideplates, the first and second sideplates including first and second elongate slots on outer surfaces thereof, the method further comprising:
extending the first and second ends of the cross strap through the first and second elongate slots before wrapping the first and second ends of the cross strap in front of the wearer's shin.
